(19) United States
(12) Patent Application Publication
(10) Pub. No.: US 2020/0368246 A1
(43) Pub. Date: Nov. 26, 2020

(54) COMBINATION THERAPY TARGETING CANCER ASSOCIATED WITH THE HEDGEHOG PATHWAY

(71) Applicant: JAEYOUNG YOON, St. Louis, MO (US)

(72) Inventor: JAEYOUNG YOON, St. Louis, MO (US)

(21) Appl. No.: 16/764,773

(22) PCT Filed: Nov. 19, 2018
(86) PCT No.: PCT/US2018/061819

§ 371 Date: May 15, 2020

Related U.S. Application Data
(60) Provisional application No. 62/587,998, filed on Nov. 17, 2017.

(51) Int. Cl. A61K 31/5377; A61K 31/4418; A61K 31/4196; A61P 35/00

(52) U.S. Cl. CPC A61K 31/5377; A61P 35/00; A61K 31/4196; A61K 31/4418

(57) ABSTRACT

Disclosed are methods of treating a cancer associated with the hedgehog (Hh) pathway are described, as are methods of overcoming resistance to single therapy treatment of a cancer associated with hedgehog (Hh) pathway, and methods of rescuing treatment of a cancer resistant to single therapy treatment, using at least two antineoplastic agents targeting the Hh pathway.

### RELATED APPLICATIONS AND INCORPORATION BY REFERENCE

This application claims priority to U.S. provisional patent application 62/587,998, entitled COMBINATION THERAPY TARGETING CANCER ASSOCIATED WITH THE HEDGEHOG PATHWAY, filed on Nov. 17, 2017, the entire disclosure of which is hereby incorporated by reference.

### FIELD OF THE INVENTION

The present disclosure provides methods of treating a cancer associated with the hedgehog (Hh) pathway, and methods of overcoming resistance to a monotherapy in treating cancer associated with the hedgehog (Hh) pathway.

### BACKGROUND OF THE INVENTION

The hedgehog (Hh) pathway is a cellular signal transduction mechanism that is vital during embryogenesis and fetal development. The main components include a series of ligands including Sonic Hedgehog (SHH); a receptor, Patched (PTCH); a transmembrane protein, Smoothened (SMO); and gene expression regulators, glioma associated oncogene transcription factors (GLI1, GLI2, GLI3). In a healthy mature state, this pathway is inhibited and quiescent.

When this pathway becomes dysregulated or reactivated, cellular proliferation occurs. The Hh pathway is abnormally active in human cancers including tumors of the skin, brain, muscle, gastrointestinal tract, breast, pancreas and prostate. A link between basal cell carcinoma and dysregulation of Hh pathway was discovered by studying families with Basal Cell Nevus Syndrome (Gorlin's Syndrome). These patients sometimes develop hundreds of basal cell cancers over a lifetime.

Certain drugs that target and inhibit the Hh pathway are used treat basal cell carcinomas. In particular, the SMO protein has been a target. Erivedge® (vismodegib; Genentech) was approved by the FDA in early 2012 for use in advanced or inoperable basal cell carcinomas. Clinical studies showed dramatic effects in reducing tumor burden on about half the patients. Odomzo® (sonidegib; Sun Pharmaceuticals) was approved in 2014 and showed a clinical response profile similar to Erivedge®. Both of these ligands target the SMO protein and inactivate it, repressing the expression of proto-oncogenic genes. The exact location of contact of each of these drug molecules with SMO is not known but both are thought to be in a similar area or pocket of SMO. The recommended, i.e., therapeutically effective dose for each of Odomzo® and Erivedge® produce a range of side effects. Additionally, in many patients Odomzo® and Erivedge® therapy is either not effective (primary resistance), or results in development of secondary resistance after an initial period of treatment. Still further, resistance developed to either of Odomzo® and Erivedge® appears to confer resistance to the other.

A long-felt need thus remains for improved therapy for cancers associated with the Hh cellular signal transduction pathway.

### SUMMARY OF THE INVENTION

One aspect of the present disclosure encompasses a method of treating a cancer associated with a hedgehog (Hh) pathway in a subject in need thereof, the method comprising administering to the subject a therapeutically effective amount of a first antineoplastic agent targeting a component of the Hh pathway, and a therapeutically effective amount of a second antineoplastic agent targeting a component of the Hh pathway. In one aspect, the component of the Hh pathway targeted by the first antineoplastic agent is a component of a first mechanism in the Hh pathway, and the component of the Hh pathway targeted by the second antineoplastic agent is a component of a second mechanism in the Hh pathway, wherein the first mechanism and the second mechanism in the Hh pathway are different. The at least two antineoplastic agents may each target a different mechanism by targeting a different component of the Hh pathway, or alternatively, each of the at least two antineoplastic agents target the same one component of the Hh pathway, but by a different mechanism.

A component of the Hh pathway may be selected from Sonic Hedgehog (SHH), a receptor, Patched (PTCH1 and PTCH2), a transmembrane protein, Smoothened (SMO) and gene expression regulators, glioma associated oncogene transcription factors (GLI1, GLI2, GLI3), growth arrest specific protein 1 (GAS1), Cell Adhesion Associated, Oncogene Regulated (CDON), Brother of CDO (BOC), SUFU Negative Regulator Of Hedgehog Signaling (SUFU), Kinesin Family Member 7 (Kif7), hedgehog-interacting protein (HHIP1), and Speckle-Type POZ Protein (SPOP). In one aspect, each of the at least two antineoplastic agents target Smoothened protein of the Hh pathway, but each targets SMO by a different mechanism.

The at least two antineoplastic agents may be selected from the group consisting of GDC-0449 (Vismodegib/Erivedge®), Odomzo®^{®} (sonidegib, LDE225, Erismodegib), Erivedge® (vismodegib), BMS-833923/XL139, PF-04449913 (Glasdegib), LY2940680 (Taladegib), IPI-926 (Saridegib), Arsenic Trioxide (ATO), Cyclopamine, CUR61414, PF-5274857, TAK-441, MRT-92, Jervine, GANTs, RU-SK/43-129/130, Shh Monoclonal Antibody 5E1-135, a triazole antifungal agent, and any combination of any thereof. In one aspect, one of the at least two antineoplastic agents is vismodegib (Erivedge®), or sonidegib (Odomzo®), or a combination thereof. In another aspect, one of the at least two antineoplastic agents is a triazole antifungal agent. When one of the at least two antineoplastic agents is a triazole antifungal agent, the agent may be selected from itraconazole, fluconazole, posaconazole, voriconazole and any combination thereof. In another aspect, one of the at least two antineoplastic agents is itraconazole, and another of the at least two antineoplastic agents is vismodegib (Erivedge®), or sonidegib (Odomzo®), or a combination thereof.

When the at least two antineoplastic agents are itraconazole and sonidegib, or itraconazole and vismodegib, the itraconazole can be administered, for example, at a dosage of about 200 mg per day for 2 weeks out of a month and the sonidegib or vismodegib is administered at a dosage of about 100 mg, about 150 mg, or about 200 mg per day, daily or every other day. Alternatively, itraconazole may be administered at a dosage of about 100 mg per day for 2 weeks out of a month and the sonidegib or vismodegib is administered at a dosage of about 100 mg, about 150 mg, or about 200 mg per day, daily or every other day. Additionally, itraconazole may be administered for about 8 months at a dosage of about 100 mg per day for 2 weeks out of a month and sonidegib or vismodegib may be administered at a dosage of about 100 mg per day, followed by a period of administration of itraconazole of about 100 mg per day for 2 weeks out of a month and administration of sonidegib or vismodegib at a dosage of about 100 mg every other day.

The cancer may be selected from the group consisting of tumors of skin, brain, muscle, gastrointestinal tract, breast, pancreas and prostate. Additionally, the cancer may be a cancer located beyond the blood brain barrier and/or may be located within the cranium or the spinal cord. In some embodiments, the cancer is basal cell carcinoma, advanced/metastatic basal cell carcinoma, or medulloblastoma. Further, the cancer may be resistant to vismodegib, the subject may be unresponsive to sonidegib, or the subject may be not capable of tolerating vismodegib and/or sonidegib.

In any of the methods, the therapeutically effective dose of each of the least two antineoplastic agents may be reduced when compared to the therapeutically effective dose of each antineoplastic agent when required for monotherapy. In any of the methods, the subject may have demonstrated primary or secondary (drug-induced) resistance to one of the antineoplastic agents. For example, the subject may have demonstrated primary or secondary resistance to sonidegib or vismodegib when administered as a monotherapy. In any of the methods, the subject may have exhibited side effects when administered a therapeutically effective dose of an antineoplastic agents such as vismodegib and/or sonidegib when administered as a monotherapy. In any of the methods, a therapeutically effective dose of each of the at least two antineoplastic agents may be reduced relative to the therapeutically effective dose of each antineoplastic agent when administered individually.

Another aspect of the present disclosure encompasses a method of overcoming resistance to single therapeutic agent used for treatment of cancer associated with hedgehog (Hh) pathway. The method comprises administering to a subject in need thereof an antineoplastic therapy comprising a first antineoplastic agent and a second antineoplastic agent each targeting the Hh pathway. The cancer is resistant to the first antineoplastic agent, and each of the two antineoplastic agents targets a different mechanism of the Hh pathway. The cancer and subject may be any of those as described herein.

A further aspect of the present disclosure encompasses a method of rescuing treatment of a cancer associated with hedgehog (Hh) pathway experienced with single therapy treatment using a first antineoplastic agent targeting a Hh pathway. The method comprises administering to a subject in need thereof an antineoplastic therapy comprising at least two antineoplastic agents targeting the Hh pathway, wherein the experienced cancer is no longer responsive to the single therapy treatment, wherein one of the at least two antineoplastic agents is the first antineoplastic agent, and wherein each of the at least two antineoplastic agents targets a different mechanism of the Hh pathway. The cancer and subject may be any of those as described herein.

Another aspect of the present disclosure encompasses a method of preventing development of resistance in a cancer associated with hedgehog (Hh) pathway to an antineoplastic agent. The method comprises administering to a human subject in need thereof an antineoplastic therapy comprising at least two antineoplastic agents targeting the Hh pathway. Each of the at least two antineoplastic agents targets different mechanisms of the Hh pathway. The first antineoplastic agent may be vismodegib or sonidegib. The cancer and subject may be any of those as described herein.

In any of the methods, the first antineoplastic agent can be vismodegib or sonidegib or a combination thereof, and the second antineoplastic agent can be a triazole antifungal agent such as itraconazole, fluconazole, posaconazole, voriconazole and any combination thereof. In any of the methods, the first antineoplastic agent can be vismodegib or sonidegib or a combination thereof, and the second antineoplastic agent can be itraconazole.

### BRIEF DESCRIPTION OF THE FIGURES

FIGS. 1 A-L shows paired colored photographs of twelve (12) basal cell carcinoma patients before and after combined treatment with itraconazole and Odomzo® and/or Erivedge®. The left column shows patients at pre-treatment; right column shows patients at several weeks after start of treatment.

FIGS. 2A and 2B depict advanced Basal Cell Carcinoma of the frontal lobe monitored by MRI imaging of a single patient during combination therapy with sonidegib and itraconazole. FIG. 2A depicts T2 weighted axial images of the brain, face and orbits demonstrating regression of the intracranial lesion over time. FIG. 2B depicts T2 weighted sagittal images of the brain, face and orbits demonstrating regression of the intracranial lesion over time. Arrow indicates the lesion within the frontal lobe.

### DETAILED DESCRIPTION

Although both clinical trials for Erivedge® and Odomzo® have shown dramatic initial clinical results, several problems arise when these drugs are administered as monotherapy (i.e., when administered as the only antineoplastic therapy):
Low response rate: Less than half of treated patients respond to therapy. The Erivedge® response rate was 43% for advanced basal cell carcinoma and 30% for metastatic basal cell carcinoma. The Odomzo® response rate was 47% for advanced basal cell carcinoma and only 17.4% metastatic tumors. In addition, the number of patients on long term therapy continued to decline with time from either poor drug tolerance by the patient or the development of tumor resistance. Currently, very few patients continue long term on Erivedge®.
Drug Resistance: A significant number of patients develop drug resistance within the first year. This means that a cure or long-term therapy is not an option for most of the patients placed on either of these medications alone. In addition, once a patient develops resistance to Erivedge®, he/she also becomes resistant to Odomzo® as well, which limits the therapeutic options for these patients. Resistance to sonidegib is also thought to confer resistance to vismodegib because the binding or active sites of the drugs appear to be near each other. The amino acid mutations are thought to cause conformational changes that affect both drugs.
Poor Patient Tolerance: Many side effects of both drugs were observed in their clinical trials. Some side effects were so severe that treatment had to be stopped. In the Erivedge® trial: 33% of patients experienced adverse side effects such as muscle spasms, alopecia, taste loss, weight loss and fatigue; 25% showed severe grade 3 or 4 adverse reactions. In the Odomzo® trial: 32% of patients experienced adverse side effects, with 22% having to discontinue treatment due to toxic effects.

The present disclosure provides compositions and methods for addressing the foregoing challenges, and is based in part on surprising results obtained from the combination therapies described herein.

With regard to the Hh pathway and cancers associated with it, it is believed that the present disclosure provides the very first evidence that multiple hits on the Hh pathway using the combination therapy described herein works much better than predicted from the sum of effects of each component agent when administered as monotherapy. Put differently, the results are surprisingly greater than the “sum of the parts.” Further, it is believed that the present disclosure provides the very first evidence of a “rescue effect” of combination therapy as described herein, i.e., that the combination therapy can reverse or overcome resistance of a cancer to treatment with a particular therapeutic agent, which may have been previously administered to the cancer patient as a monotherapy.

Accordingly, the present disclosure is based in part on the surprising discovery that certain combination therapies targeting the Hh pathway have a more than merely additive result in improving the treatment results for a cancer associated with hedgehog (Hh) signaling pathway, as compared to treatment using a single therapeutic agent to target the cancer associated with the Hh pathway. More specifically, it was surprisingly discovered that the combination cancer therapy described herein, which targets the Hh pathway, produces unexpectedly improved response rate to the treatment, markedly improves patient drug tolerance over monotherapy, and surprisingly reverses, overcomes or reduces development of resistance to those therapeutic agents that have been known to induce cancer resistance when used in single agent therapy. Additionally, the combination therapy surprisingly allows for use of an unexpectedly lower dose of each therapeutic agent for treating the cancer as compared to the dose of each agent required when administered as a monotherapy. Significantly, it was especially surprising that the combination therapy is so effective in treating a cancer in a patient, even when that cancer is demonstrably resistant to one of the therapeutic agents when administered to the patient as a single agent (monotherapy). In other words, the combination therapy as described herein is unexpectedly capable of overcoming and rescuing susceptibility of a cancer to treatment with a given therapeutic agent, even when that cancer is demonstrably resistant to treatment using that given therapeutic agent alone (in monotherapy), and when the combination therapy includes that given therapeutic agent to which the cancer is resistant.

Two lines of evidence support an augmented or synergistic clinical effect by administering two or more hedgehog inhibitors in combination. First, in phase III clinical trials for Odomzo® and Erivedge®, a successful response to treatment was measured by a reduction of tumor burden by greater than 30%. In both trials, approximately 40%-50% of patients responded to therapy. In contrast, as detailed further in the Examples below, the inventor has achieved a surprisingly higher response rate of about 90% (15 out of 16 patients) of patients showing at least 30% improvement within 5-6 weeks. Response was measured by improvement of symptoms, decreased tumor thickness on palpation, improvement in clinical appearance, measurement of tumor, or imaging using MRI scan. Two patients had post treatment biopsies and showed no residual tumor and thus had a complete response to treatment. Although the response criteria are slightly different than those used in the clinical trials for Odomzo® and Erivedge® monotherapy, a response rate of 90% is a dramatic improvement as compared to those obtained with monotherapy using either drug alone.

The second observation that supports an augmented or synergistic clinical effect by administering two or more hedgehog inhibitors in combination as disclosed herein is that hedgehog inhibitors as described herein are effective at dosages significantly lower than those recommended for the drug when administered as the approved monotherapy. For example, the recommended dosage for Odomzo® is 200 mg daily, and for Erivedge® is 150 mg daily. In contrast, when combined with at least one additional (i.e., a second), different hedgehog inhibitor, such as but not limited to a triazole antifungal such as itraconazole, the therapeutically effective dose of Odomzo® and Erivedge® is reduced by about 60% to about 70%. When the second hedgehog inhibitor used is itraconazole, clinical efficacy correlates with increasing dose. Further, when used alone, itraconazole is most effective when used at a dose of greater than about 400 mg per day, which can increase the risk and severity of side effects. In contrast, when itraconazole is administered in combination with Odomzo® or Erivedge® according to the present disclosure, the therapeutically effective dose of itraconazole is reduced to as low as 100 mg per day in some instances. In other aspects, the lowest therapeutically effective dose of itraconazole is lowered to 100 mg per day or 200 mg per day, when itraconazole is administered in combination therapy according to the present disclosure with another hedgehog inhibitor. The hedgehog inhibitor can be for example Odomzo® 200 mg, and/or Erivedge® 150 mg, 2-3 times per week (in either case, about 29% −43% of recommended dose).

The side effect profiles of Odomzo® and Erivedge® are similar to one another. Ninety-five to one hundred percent of patients treated with Odomzo® and Erivedge® monotherapy at the respective recommended doses experience treatment-emergent adverse events. The most common side effects are muscle cramps, fatigue, hair loss, taste changes, weight loss, diarrhea and constipation. Although these are not life threatening, they lead to decreased quality of life and for many patients to terminate treatment. It remains one of the biggest reasons why these medications are not used more widely. In contrast, the combination therapy described herein risks only those side effects commensurate with the relatively reduced dose of Odomzo® and/or Erivedge® used in the combination therapy. Further, some patients administered the combination therapy disclosed herein, using a dose of either Odomzo® and/or Erivedge® that was less than 50% of the recommended dose of the drug when administered as a monotherapy, had no side effects at all, which is extremely unusual. Overall, the amount of Odomzo® and/or Erivedge® administered in the combination therapy appears to correlate with the severity and/or the number of adverse events. The decrease in intensity and frequency of side effects from the combination therapy as disclosed herein will make treatment more tolerable and comfortable for patients and encourage them to stay on medication until the end of treatment regimen.

Additionally, the combination therapy disclosed herein appears to present little risk to internal organs and metabolic heath. Patients were typically followed on a weekly basis with clinical exam, interview and laboratory testing. Labs included test to monitor for the liver, kidney, electrolytes, muscle breakdown, pancreas, and blood counts (chemistry panel, liver function test, amylase, total creatinine kinase, complete blood count), well beyond the testing recommended by the suppliers of Odomzo® and Erivedge®. Not a single laboratory abnormality was observed in any patient during or after the combination therapy as disclosed herein, using itraconazole in combination with Odomzo® and/or Erivedge®. In some patients, all three of itraconazole, Odomzo® and Erivedge® were used in combination, with no evidence of toxicity or side effects. The age range of the entire group was 19 years to approximately 90 years old. Two patients presented with side effects sufficiently severe that the combination therapy was discontinued, however this result is merely consistent with the risk of side effects in patients treated by monotherapy with Odomzo® and/or Erivedge®. When all treatment weeks are combined among patients tried with the combination therapy disclosed herein, the result was surprising: well over 200 treatment-weeks without a single significant incident distinguishable from the side effects observed when Odomzo® and/or Erivedge® are administered as monotherapy.

The combination therapy described herein also appears to reduce drug induced resistance. Reports indicate that patients who become resistant to either Odomzo® or Erivedge®, also become resistant to the other (Clinical Cancer Research 2016;22(6):1325-1329). The first patient to receive combination therapy as disclosed herein had a basal cell carcinoma that had advanced from the nose, through the sinus and into the right frontal lobe of the brain. Initially treated with Erivedge® in a clinical trial several years previous, then later with a second round, the patient developed resistance to Erivedge® and the basal cell carcinoma continued to grow through treatment at which time treatment was terminated. The patient was then treated with combination therapy as disclosed herein, and the Erivedge® resistant tumor responded dramatically and disappeared from the brain within three (3) months. The patient continued on varying doses of combination therapy, and serial imaging scans continued to show resolution within the brain for approximately one year until the patient's death from a heart attack. This suggests that Erivedge® or Odomzo® induced resistance may be overcome by using alternate hedgehog inhibitors in combination. Secondly, when basal cell carcinoma patients are treated with either Odomzo® or Erivedge®, some tumors are initially resistant to treatment, i.e., exhibit primary resistance. Some tumors are initially responsive, but then later become resistant to treatment and start to grow again during therapy, i.e., exhibit secondary resistance, or drug induced resistance. Secondary resistance has been observed in 21% of treated patients and the mean time for secondary resistance is about 52 weeks of treatment. In contrast, no patients treated with the combination therapy as disclosed herein developed drug acquired or secondary resistance to therapy as of the date of the present disclosure. In addition, that 90% of patients treated with the combination therapy as described herein were responsive to treatment, as opposed to 40%-50% of patients treated with monotherapy, shows a lower rate of primary resistance.

### I. Method of Treating

Accordingly, in one aspect, the present disclosure provides a method of treating a cancer associated with hedgehog (Hh) pathway in a subject in need thereof, such as a human patient. In one aspect, the method comprises administering to the subject in need thereof an antineoplastic therapy comprising at least two antineoplastic agents targeting the Hh pathway, wherein each of the at least two antineoplastic agents target the Hh pathway by targeting different proteins of the Hh pathway.

Each of the at least two antineoplastic agents targets a different mechanism of the Hh pathway. Each of the at least two antineoplastic agents may target a different mechanism of the Hh pathway by targeting at least two different components of the Hh pathway. Alternatively, each of the at least two antineoplastic agents may target the same component of the Hh pathway, but by a different mechanism.

(a) Hedgehog Pathway Components

Any of the components of the Hh pathway may be targeted, provided the at least two antineoplastic agents target the Hh pathway at different components of the pathway, or by different mechanisms. Non-limiting examples of components of the Hh pathway include Sonic Hedgehog (SHH), a receptor, Patched (PTCH1 and PTCH2), a transmembrane protein, Smoothened (SMO) and gene expression regulators, glioma associated oncogene transcription factors (GLI1, GLI2, GLI3), growth arrest specific protein 1 (GAS1), Cell Adhesion Associated, Oncogene Regulated (CDON), Brother of CDO (BOC), SUFU Negative Regulator Of Hedgehog Signaling (SUFU), Kinesin Family Member 7 (Kif7), hedgehog-interacting protein (HHIP1), and Speckle-Type POZ Protein (SPOP).

In one aspect, at least one of the antineoplastic agents targets the SMO protein. In another aspect, each of the at least two antineoplastic agents target the SMO protein of the Hh pathway, but each by a different mechanism.

(b) Subject

Suitable subjects may include, without limit, humans, as well as companion animals such as cats, dogs, rodents, and horses; research animals such as rabbits, sheep, pigs, dogs, primates, mice, rats and other rodents; agricultural animals such as cows, cattle, pigs, goats, sheep, horses, deer, chickens and other fowl; zoo animals; and non-human primates such as chimpanzees, monkeys, and gorillas. The subject can be any age without limitation. In one aspect, the subject is a human adult, such as a human cancer patient.

(c) Cancer

Methods and combinations therapies described herein may be used to treat any neoplasm or cancer associated with the Hh pathway. The neoplasm may be malignant or benign; the cancer may be primary or metastatic; and the neoplasm or cancer may be early stage or late stage. Importantly, unlike many other cancer drugs, combination therapy as described herein, such as, e.g., itraconazole in combination with Odomzo®, can penetrate the blood brain barrier even when administered orally. As such, the methods and combination therapies described herein can even be used to treat cancers within the cranium and spinal cord which are protected by the blood brain barrier.

Non-limiting examples of neoplasms or cancers that may be treated according to the present disclosure include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood).

In one aspect, the cancer is Basal Cell Carcinoma, Advanced/Metastatic Basal Cell Carcinoma, Basal Cell Nevus Syndrome, Medulloblastoma, Recurrent Medulloblastoma, Metastatic Castration-Resistant Prostate Cancer, Chondrosarcoma, Advanced Pancreatic Cancer, Metastatic Pancreatic Cancer, Myelofibrosis, Metastatic Gastric & Esophageal Cancer, Advanced Prostate Adenocarcinoma, Small-Cell Lung Cancer, Keratocystic Odontogenic Tumor, Advanced Solid Tumors, Acute Myeloid Leukemia, Intracranial Meningioma, Prostate Cancer, Castration-Resistant Prostate Cancer, Pancreatic Adenocarcinoma, Advanced/Metastatic Pancreatic Cancer, Refractory Multiple Myeloma, Recurrent Ovarian Cancer, Triple-Negative Breast Cancer, Myeloid Malignancies Basal Cell Carcinoma, Advanced Gastroesophageal Adenocarcinoma, Advanced/Metastatic Hepatocellular Carcinoma, Relapsed Medulloblastoma, Chronic Myeloid Leukemia, Chronic Myelomonocytic Leukemia, Myelodysplastic Syndrome, Esophageal Cancer, Advanced Pancreatic Adenocarcinoma, Non-Small-Cell Lung Cancer, Malignant Glioma, Acute Promyelocytic Leukemia, and Chronic Myelogenous Leukemia. More preferably, the cancer is Basal Cell Carcinoma or Advanced/Metastatic Basal Cell Carcinoma.

A cancer or neoplasm may be a cancer that has not previously been treated. Alternatively, a cancer may be resistant to single agent therapy. For instance, a cancer that can be treated using a method of the invention can be one that has shown resistance to Odomzo® (sonidegib), Erivedge® (vismodegib), Arsenic Trioxide (ATO), or combinations thereof.

(d) Antineoplastic Agents

Any therapeutic antineoplastic agent with activity against a component of the Hh pathway can be used in a method of the invention. Non-limiting examples of therapeutic agents with activity against a component of the Hh pathway include GDC-0449 (vismodegib/Erivedge®), Odomzo® (sonidegib, LDE225, erismodegib), BMS-833923/XL139, PF-04449913 (glasdegib), LY2940680 (taladegib), IPI-926 (saridegib), Arsenic Trioxide (ATO), Cyclopamine, CUR61414, PF-5274857, TAK-441, MRT-92, Jervine, GANTs, RU-SK/43-129/130, Shh Monoclonal Antibody 5E1-135, and a triazole antifungal agent. It will be apparent to a skilled artisan that other agents that are yet to be shown to have activity against a component of the Hh pathway can also be used in methods of the invention.

In one aspect, one of the at least two antineoplastic agents is a triazole antifungal agent. Non-limiting examples of triazole antifungal agents include fluconazole, itraconazole, posaconazole, and voriconazole, and any combination thereof. In one aspect, the triazole antifungal agent is itraconazole. In another aspect, a combination of therapeutic agents according to the present disclosure is itraconazole and Odomzo® (sonidegib). In another aspect, a combination of therapeutic agents according to the present disclosure is itraconazole and Erivedge® (vismodegib). In another aspect, a combination of therapeutic agents according to the present disclosure is itraconazole, Odomzo® and Erivedge®. In yet another aspect, a combination of therapeutic agents according to the present disclosure is itraconazole and ATO; or itraconazole, ATO, Odomzo® and/or Erivedge®. Other combinations include, without limitation:

fluconazole+Odomzo®

fluconazole+Erivedge®

fluconazole+Odomzo®+Erivedge®

posaconazole+Odomzo®

posaconazole+Erivedge®

posaconazole+Odomzo®+Erivedge®

voriconazole+Odomzo®

voriconazole+Erivedge®

voriconazole+Odomzo®+Erivedge®

In any of the above combinations, it should be understood at minimum that any triazole antifungal agent can be substituted for the itraconazole, fluconazole, posaconazole, or voriconazole. Further, it should be understood that any therapeutic agents with activity against a component of the Hh pathway, such as BMS-833923/XL139, PF-04449913 (glasdegib), LY2940680 (taladegib),IPI-926 (saridegib), Arsenic Trioxide (ATO), Cyclopamine, CUR61414, PF-5274857, TAK-441, MRT-92, Jervine, GANTs, RU-SK/43-129/130, and Shh Monoclonal Antibody 5E1-135, can be substituted in any of the above combinations for Odomzo® or Erivedge® in any combination.

(e) Dosage Regimen

Each therapeutic agent in a combination therapy of the invention can be administered orally or parenterally, including the intravenous, intramuscular, intraperitoneal, subcutaneous, rectal, topical, and transdermal routes of administration. Methods of formulating therapeutic agents for each route of administration are well known in the art.

Generally, the therapeutic agents will be administered in a therapeutically effective amount which includes prophylactic amounts or lower dosages for example, when combined with another agent. As used herein, “therapeutically effective amount” refers to doses of compound sufficient to provide circulating concentrations high enough to impart a beneficial therapeutic effect on the recipient thereof. The precise amount to be administered can be determined by the skilled practitioner in view of desired dosages, side effects, medical history of the patient, and observation of a patient's response to therapy as described herein.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

At least one of the therapeutic agents in the combination therapy can be administered using the same dosage regimen (dose, frequency, and duration of treatment) that is typically employed when the agent is used as monotherapy for treating the same cancer. Alternatively, the patient receives a lower total amount of at least one, or both, of the therapeutic agents in the combination therapy than when the agent is used as monotherapy, e.g., smaller doses, less frequent doses, and/or shorter treatment duration.

A combination therapy of the invention may be used prior to or following surgery to remove a tumor and may be used prior to, during or after radiation therapy.

A combination therapy of the invention may be administered to a patient who has not been previously treated with a biotherapeutic or chemotherapeutic agent, i.e., is treatment-naïve. Alternatively, the combination therapy is administered to a patient who failed to achieve a sustained response after prior therapy with surgery, a biotherapeutic, or chemotherapeutic agent, i.e., is treatment-experienced.

Selecting a dosage regimen (also referred to herein as an administration regimen) for a combination therapy of the invention can and will vary depending on several factors, including the therapeutic agent, the serum or tissue turnover rate of the agent, the cancer to be treated, the level of symptoms, the immunogenicity of the entity, and the accessibility of the target cells, tissue or organ in the individual being treated, the individual, and the physical condition of the subject, among other variables. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (Nov. 2002). Determination of the appropriate dosage regimen may be made by the clinician, e.g., using parameters or factors known or suspected in the art to affect treatment or predicted to affect treatment, and will depend, for example, on the patient's clinical history (e.g., previous therapy), the type and stage of the cancer to be treated and biomarkers of response to one or more of the therapeutic agents in the combination therapy.

Each antineoplastic agent in a combination therapy may be administered by continuous infusion or by doses at intervals of, e.g., daily, every other day, three times per week, or one time each week, two weeks, three weeks, monthly, bimonthly, etc. For example, as further detailed below in the Examples, administration of any one neoplastic agent may be QD: Daily; QOD: Every other day; once a week on any day of the week; twice a week, such as but not limited to Monday AND Friday, or Tuesday AND Thursday; or three times per week, such as but not limited to Monday AND Wednesday AND Friday. It should be understood that the present disclosure contemplates various combinations of administration schedules and combinations of neoplastic agents readily identifiable by those of skill in the art and as exemplified in non-limiting fashion by the Examples below.

When one of the at least two antineoplastic agents is itraconazole, the agent is administered at a dosage ranging from about 10 mg per day to about 500 mg per day, from about 50 mg per day to about 300 mg per day, or from about 100 mg per day to about 200 mg per day. Additionally, any does of itraconazole may be administered every day throughout the duration of treatment, or every other day during the treatment. In one aspect, itraconazole is administered at a dosage of about 100 mg per day, or at a dosage of about 200 mg per day, daily or every other day. In one aspect, itraconazole is administered every day for 2 weeks out of a month. In another aspect, itraconazole is administered every other day for 2 weeks out of a month.

When one of the at least two antineoplastic agents is Odomzo®, the agent is administered at a dosage ranging from about 10 mg per day to about 500 mg per day, from about 50 mg per day to about 300 mg per day, from about 100 mg per day to about 200 mg per day, daily or every other day. In one aspect, Odomzo® is administered at a dosage of about 200 mg per day, or at a dosage of about 100 mg per day, daily or every other day. In another aspect, Odomzo® is administered at a dosage of about 100 mg, about 150 mg, or about 200 mg per day, daily or every other day. In one aspect, any dose of Odomzo® may be administered every day throughout the duration of treatment, or every other day during the treatment.

When one of the at least two antineoplastic agents is Erivedge®, the agent is administered at a dosage ranging from about 10 mg per day to about 500 mg per day, from about 50 mg per day to about 300 mg per day, from about 100 mg per day to about 200 mg per day, daily or every other day. In another aspect, Erivedge® is administered at a dosage of about 100 mg, about 150 mg, or about 200 mg per day, daily or every other day. In one aspect, any dose of Erivedge® may be administered every day throughout the duration of treatment or every other day during the treatment.

In any of the methods, in non-limiting example, itraconazole is administered at a dose of at least about 100 mg to about 200 mg daily, and administered over the same period that Odomzo® and/or Erivedge® is administered at a frequency of twice (2 times) per week. For example, the itraconazole is administered during the same treatment period in which, for example, one pill of Erivedge® @150 mg, or one pill of Odomzo® @200 mg, twice per week, is administered. Alternative dosage regimens within the scope and spirit of the present disclosure will be appreciated by those of skill in the art, some examples of which are detailed in the Examples below.

### II. Method of Overcoming Resistance

In another aspect, the present disclosure provides a method of overcoming resistance to single therapy treatment of a cancer associated with hedgehog (Hh) pathway, the method comprising administering to a subject in need thereof an antineoplastic therapy comprising at least two antineoplastic agents targeting the Hh pathway, wherein the cancer is resistant to a first antineoplastic agent, wherein one of the at least two antineoplastic agents is the first antineoplastic agent, and wherein each of the at least two antineoplastic agents targets a different mechanism of the Hh pathway. The hedgehog pathway, the cancer, the subject, antineoplastic agents, and the dosage regimen used in the method may be as described in Section I. For example, the first antineoplastic agent may be vismodegib and/or sonidegib, and the second antineoplastic agent may be a triazole antifungal agent such as itraconazole, fluconazole, posaconazole or voriconazole, or any combination thereof.

### III. Method of Rescuing Treatment

In another aspect, the present disclosure provides a method of rescuing treatment of a cancer experienced with single therapy treatment using a first antineoplastic agent targeting the Hh pathway, the method comprising administering to a subject in need thereof an antineoplastic therapy comprising at least two antineoplastic agents targeting the Hh pathway, wherein the experienced cancer is no longer responsive to the single therapy treatment, wherein one of the at least two antineoplastic agents is the first antineoplastic agent, and wherein each of the at least two antineoplastic agents targets a different mechanism of the Hh pathway. The Hh pathway, the cancer, the subject, antineoplastic agents, and dosage regimen used in the method may be as described in Section I. For example, the first antineoplastic agent may be vismodegib and/or sonidegib, and the second antineoplastic agent may be a triazole antifungal agent such as itraconazole, fluconazole, posaconazole or voriconazole, or any combination thereof.

### IV. Method of Preventing Development of Resistance

In another aspect, the present disclosure provides preventing development of resistance in a cancer associated with hedgehog (Hh) pathway to an antineoplastic agent, the method comprising administering to a subject in need thereof an antineoplastic therapy comprising at least two antineoplastic agents targeting the Hh pathway, wherein each of the at least two antineoplastic agents targets different mechanisms of the Hh pathway. The Hh pathway, the cancer, the subject, antineoplastic agents, and dosage regimen used in the methods may be as described in Section I. For example, the first antineoplastic agent may be vismodegib and/or sonidegib, and the second antineoplastic agent may be a triazole antifungal agent such as itraconazole, fluconazole, posaconazole or voriconazole, or any combination thereof.

### Definitions

To facilitate understanding of the invention, several terms are defined below.

When introducing elements of the present disclosure, the articles “a,” “an,” “the,” and “said” are intended to mean that there are one or more of the elements. The use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms as used herein and in the claims shall include pluralities and plural terms shall include the singular.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The terms “about” or “approximately” means within an acceptable error range for the particular value as determined by one of ordinary skill in the art, which will depend in part on how the value is measured or determined, i.e., the limitations of the measurement system. For example, “about” can mean within 1 or 2 standard deviations, from the mean value. Alternatively, “about” can mean plus or minus a range of up to 20%, preferably up to 10%, more preferably up to 5%.

As used herein, the term “administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. Further, the term “treat” may be used to describe prophylaxis, amelioration, prevention or cure of a neoplasm or cancer. For instance, treatment of an existing cancer may reduce, ameliorate or altogether eliminate the cancer or neoplasm, or prevent it from worsening.

### EXAMPLES

The publications discussed above are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1

### Patient Case for Treatment of Aggressive Basal Cell Carcinoma Using Combination Sonidegib and Itraconazole Therapy

Mr. W. T. presented to the inventor approximately one year ago. Several years beforehand the patient had a large and aggressive basal cell carcinoma on his nose which was treated by surgery. His nose was reconstructed using a paramedian forehead flap. The cancer recurred and invaded deeply into his sinuses. He was no longer a surgical candidate and enrolled in a clinical trial for vismodegib. He initially experienced a dramatic response and the tumor shrank 70% within in the first several months. He withdrew from the study and an attempt was made to treat the remaining tumor with radiation therapy. This failed after he received the maximum dosage of radiation allowed. He was placed back on vismodegib for a second time but the tumor developed resistance to the drug. Mr. W. T. was then sent to Siteman Cancer Center at Washington University in St. Louis where they placed him on a new immunotherapy named Keytruda (pembrolizumab). He was treated for 3 cycles, but this also failed to show effectiveness. By this time, the basal cell carcinoma had metastasized to the right frontal lobe of the brain, which was observed as a 2-cm mass on MRI. At this point, the patient was offered hospice.

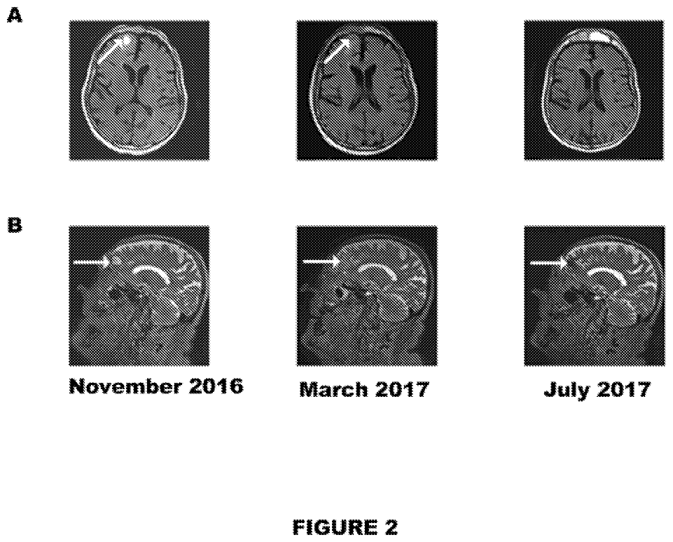

An older antifungal medicine, itraconazole, had previously been tested in a small study as monotherapy for treating basal cell carcinoma. (D. Kim et al., J Clin Oncol. 32(8):745-51 (2014 Mar. 10). Of nineteen patients with treated with itraconazole at 100 mg twice per day for one month, or 200 mg twice per day for one month, itraconazole treatment was associated with two adverse events (grade 2 fatigue and grade 4 congestive heart failure). Itraconazole reduced tumor area by 24%, and of eight patients with multiple nonbiopsied tumors, four achieved partial response, and four had stable disease. Despite the some reduction in size however, none of the BCC tumors disappeared completely with itraconazole treatment after an average of 1.1 or 2.3 months (respectively, for two different cohorts) of treatment. (Kim et al. at 749). Given W. T's prognosis, the inventor chose to try the combination of itraconazole together with sonidegib for patient W. T. Although there was no human clinical data at the start of treatment to support this use of this combination of therapeutics, the inventor felt that this was the patient's last hope and placed him on a combination of itraconazole at a dosage of 100 mg per day for 2 weeks out of a month (and 2 weeks off), together with sonidegib at 200 mg per day. Therapy was started in December of 2016. Remarkably, after 3 months, the tumor in the right frontal lobe disappeared (see FIG. 2), despite the tumor having previously developed resistance to treatment with vismodegib.

### Example 2

### Combination Therapy Using Reduced Dosage of Sonidegib and Itraconazole

Over the course of a year of treatment, the patient of Example 1 developed complicating health issues related to his heart approximately 8 months from the start of therapy. Because of drug interactions with one of his heart medications, verapamil, the dosage of sonidegib was reduced to 200 mg every other day around August of 2017. On Nov. 1, 2017, an MRI showed that even with the Odomzo® (sonidegib) dose cut in half for the last 3 months, the patient continued to be in remission with regard to his metastatic basal cell carcinoma of the brain (FIG. 2).

The patient was followed both clinically and by lab work for approximately a year of treatment with itraconazole and sonidegib. He experienced alopecia but no other major side effects even though he is close to 90 years old. Importantly, the patient is still in complete remission of his brain tumor after one year.

### Example 3

### Additional Patient Cases for Treatment of Aggressive Basal Cell Carcinoma Using Combination Therapy

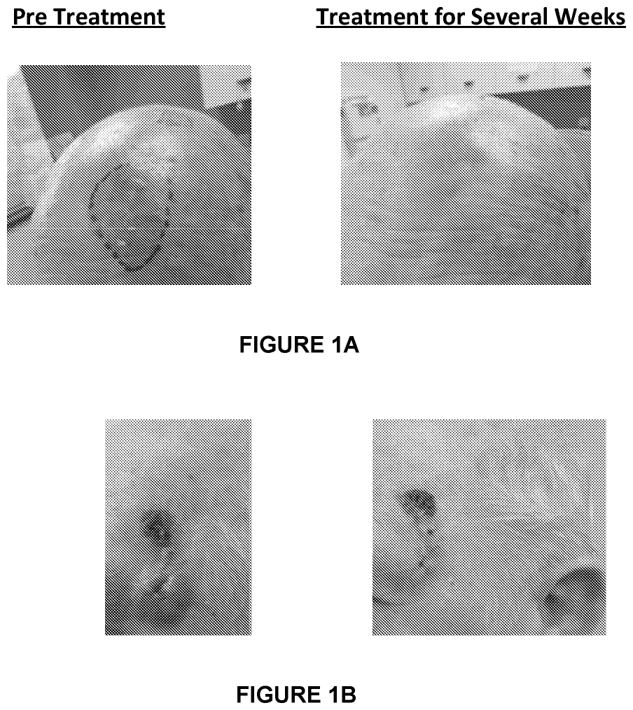

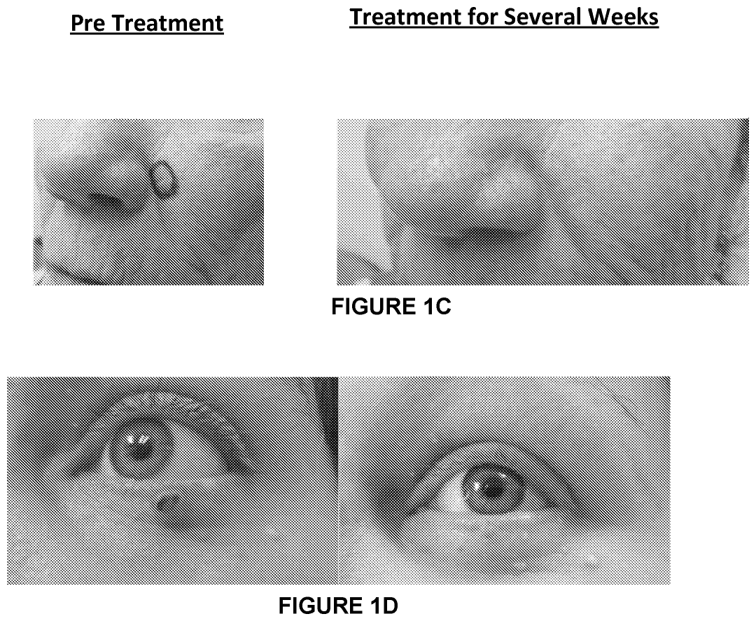

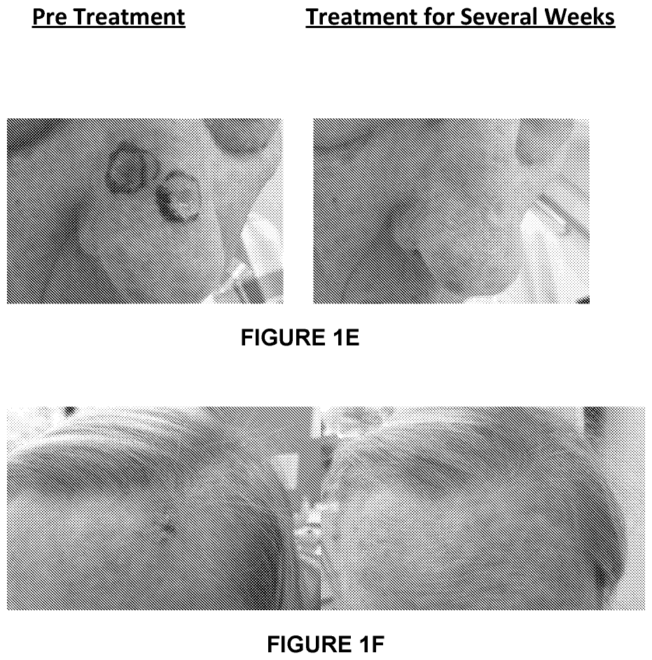

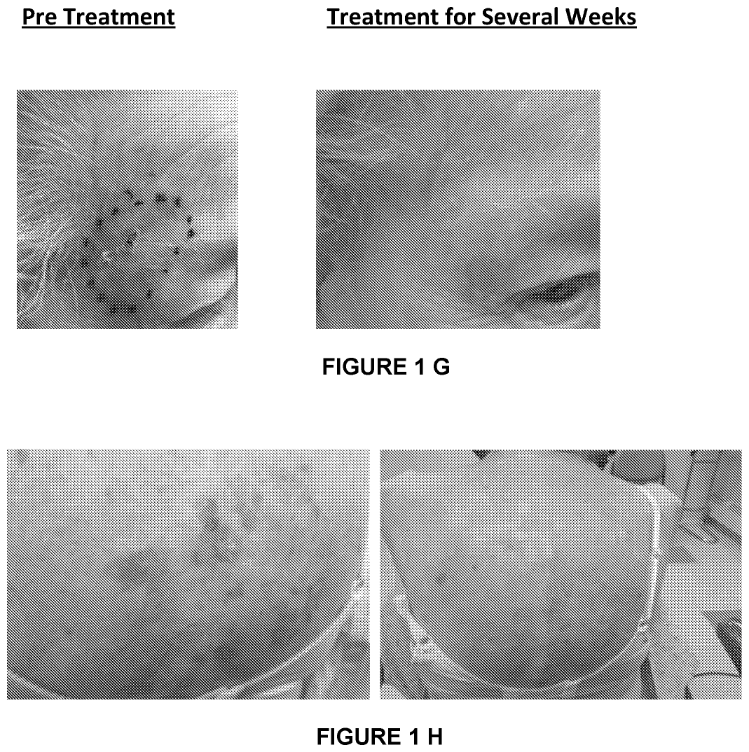

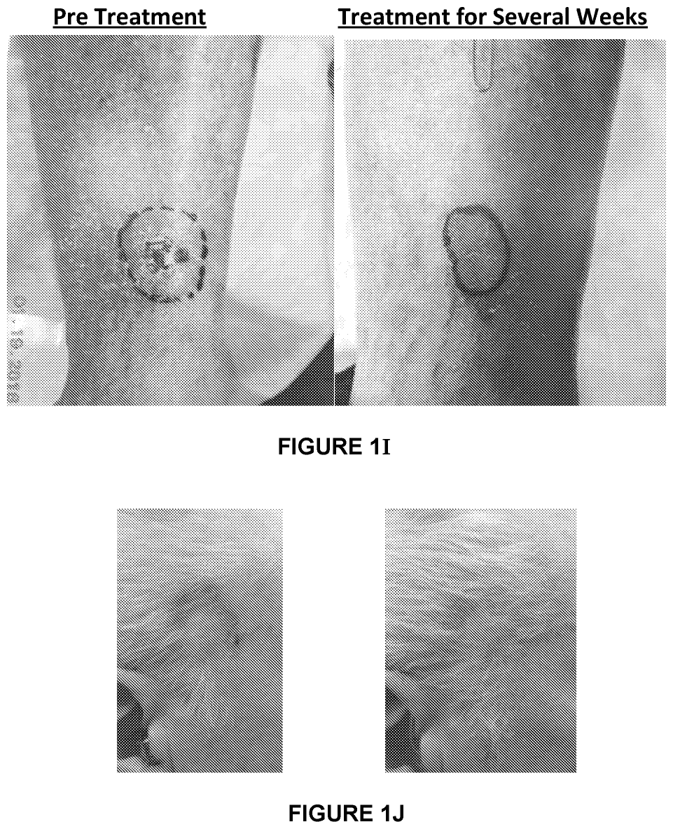

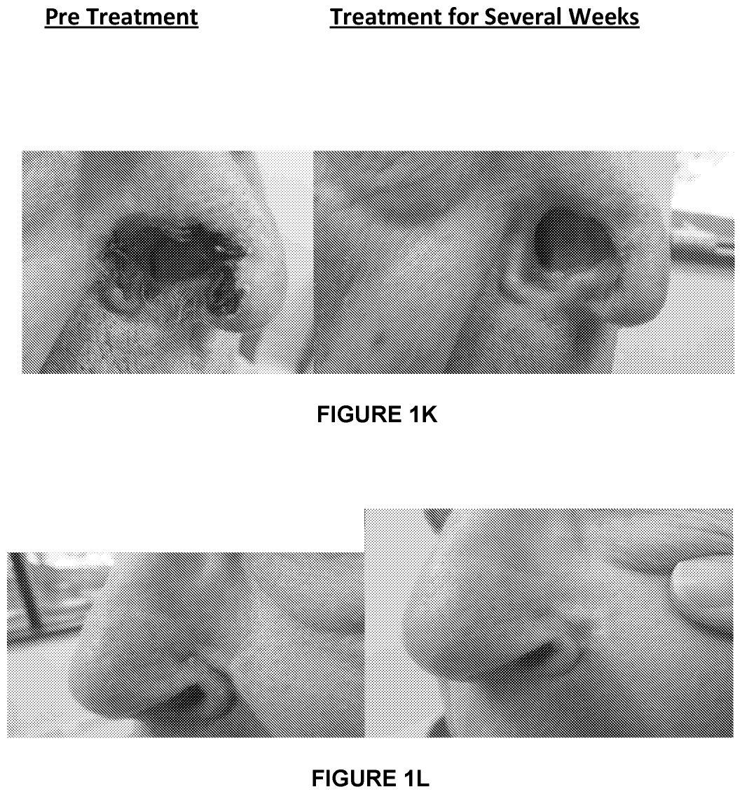

Twenty (20) patients were placed on combination therapy using itraconazole and Odomzo® and/or Erivedge®. Follow up data of at least six (6) weeks or greater for sixteen (16) patients is provided below. Photographs of pre- and post-treatment are provided in FIG. 1.

The clinical results in sixteen patients with basal cell cancer demonstrate consistent efficacy of combinations of itraconazole and Odomzo® and/or Erivedge®. FIG. 1 and Table 1 below summarize the Response of each patent, where “Response” is defined as a greater than 30% improvement as determined by an assessment of clinical appearance, measurement of tumor, improvement of symptoms, and/or thickness of tumor as determined by manual palpation, or by post treatment skin biopsy showing reduction of tumor burden or no evidence of residual cancer. Fifteen (15) patients with a greater than 5 week follow-up clearly responded to treatment. Only a single patient among those for which follow-up data was available did not show a noticeable clinical response to treatment as of the present filing.

TABLE 1

| Patient | Tumor Location | Post Treatment Biopsy | Response |
|---|---|---|---|
| Z | scalp, right canthus | Yes, no residual tumor | Yes |
| A | Left Frontal Scalp | | Yes |
| B | Left Temple, Cheek, Neck | Yes | |
| C | Left Nasolabial Fold | Yes, only superficial | Yes |
| | | tumor left | |
| D | Left Lower Eyelid Margin | Residual tumor treated | Yes |
| | | and visualized with | |
| | | Mohs surgery | |
| E | Nose, Left Helix, Scalp | | Yes |
| F | Forehead, Scalp, R Ear | | Yes |
| G | Right Eyebrow | | Yes |
| H | Back | | Yes |
| I | Left Ankle, Right Hand | | Yes |
| J | R Temporal Scalp, R Nose | | Yes |
| K | Right Nose | Residual tumor treated | Yes |
| | | and visualized with | |
| | | Mohs surgery | |
| L | Right Ala | Yes, 2 out of 3 biopsy | Yes |
| | | negative, small amount | |
| | | residual tumor left on | |
| | | 3^{rd} biopsy | |
| M | Scalp | | No |
| N | Right Frontal Lobe of Brain | | Yes |
| O | L Chest, R Arm, Back | Yes, no residual tumor | Yes |
| P | Diffuse | | Not Yet |
| Q | Face | | Not Yet |
| R | Diffuse | | Not Yet |
| S | Diffuse | | Not Yet |
| T | Face | | Awaiting Rx |
| | | | Authorization |

Table 2 below summarizes the results with respect to the range of effective doses. The recommended dose for Odomzo® when administered as a monotherapy is 200 mg per day, and for Erivedge® it is 150 mg per day. In Table 2, the “% Recommended Dose” is the total Odomzo® or Erivedge® dose given during the treatment period, expressed as a percentage of the respective recommended dose. For example: if a given dose of Odomzo® was given every other day, QOD, for 10 days, when the recommended dose Odomzo® is the same given dose but given every day, QD, then the “% Recommended Dose” as reported in Table 2 would be 50%.

The results show efficacy of treatment at dramatically reduced dosages of Odomzo® or Erivedge® when administered in combination with that itraconazole. Without intending to be bound by theory, there appears to be a synergy in clinical effect when the two different hedgehog pathway inhibitors are used in combination. To obtain the synergy, the required dose of itraconazole is at least about 100 mg to about 200 mg daily, administered over the same period that either Odomzo® OR Erivedge® is administered at a frequency of twice (2 times) per week. The itraconazole is administered during the same treatment period in which, for example, one pill of Erivedge® @150 mg, or one pill of Odomzo® @200 mg, twice per week can be used.

TABLE 2

| Patient | Tumor Response | Dose | % Recommended Dose |
|---|---|---|---|
| Z | Yes | E QD x 5 weeks | 72.6% |
| | | E QOD x 6 weeks | |
| B | Yes | O M-W-F x 4 weeks | 35.5% |
| C | Yes | O QD x 3 weeks | 62.5% |
| | | O QOD x 9 weeks | |
| D | Yes | O QD x 21 weeks | 100% |
| E | Yes | E M-W-F x 4 weeks | 43% |
| F | Yes | E M-F x 4 weeks | 29% |
| G | Yes | E QD x 7 weeks | 100% |
| A | Yes | E T-Th, O M-W x 10 wks | 49% |
| | | O M-F x 4 weeks | |
| H | Yes | O QD x 17 weeks | 100% |
| I | Yes | E QD x 8 weeks | 90% |
| | | E QOD x 2 weeks | |
| J | Yes | E QD x 6 weeks | 100% |
| K | Yes | E QD x 11 weeks | 100% |
| L | Yes | O QD x 16 weeks | 100% |
| M | Yes | O QD x 12 weeks | 100% |
| N | Yes | O QD x 2 weeks | 48.6% |
| | | O QOD x 4 weeks | |
| | | O M-W-F x 18 weeks | |

Abbreviations:
O: Odomzo, sonidegib
E: Erivedge, vismodegib
QD: Daily
QOD: Every other day
M: Monday
T: Tuesday
W: Wednesday
Th: Thursday
F: Friday
M-F: Monday AND Friday
T-Th: Tuesday AND Thursday
M-W-F: Monday AND Wednesday AND Friday

The resulting efficacy of Odomzo® or Erivedge® at significantly reduced dosages when combined with an antifungal such itraconazole also significantly reduces the risk and severity of side effects. Table 3 below summarizes the side effect profile for each patient treated with the “% Recommended Dose” as reported in Table 2 for that patient. The most common side effects among all patients were fatigue, hair loss, muscle cramping, taste changes, diarrhea, weight loss. Two patients terminated treatment because of side effects. Three patients had no side effects. In general, the number, frequency and severity of side effects correlated with the % of recommended dose.

TABLE 3

| Patient | % Recommended Dose | Lab Changes | Side Effects |
|---|---|---|---|
| Z | 72.6% | None | Mild Leg Cramping |
| E | 43% | None | None |
| C | 62.5% | None | Mild Leg Cramping |
| D | 100% | None | Significant fatigue, |
| | | | severe hair loss |
| | | | muscle cramps |
| B | 35.5% | None | None |
| F | 29% | None | Moderate muscle |
| | | | cramping |
| | | | Mild diarrhea |
| G | 100% | None | Fatigue, hair loss, leg |
| | | | cramps |
| | | | Taste changes |
| A | 49% | None | Moderate leg cramping, |
| | | | Constipation/Diarrhea |
| H | 100% | None | Severe fatigue, hair |
| loss, | | | |
| I | 90% | None | severe diarrhea |
| | | | Discontinued medication |
| J | 100% | None | Fatigue, depression, |
| hair | | | loss |
| | | | Taste changes |
| | | | Discontinued medication |
| K | 100% | None | Fatigue, hair loss, |
| | | | cramping, weight loss |
| | | | (7 lbs.) |
| L | 100% | None | None |
| N | 100% | None | Hair loss, taste changes, |
| | | | Fatigue, weight loss |
| O | 48.6% | None | Severe leg cramping |
| | | | requiring medication |
| | | | decrease, hair loss, |
| | | | constipation. Pt notes |
| | | | severity of cramping |
| | | | correlated with dosing. |
| M | 49% | None | Leg cramping, mild |
| | | | fatigue, hair loss |

Abbreviations:
O: Odomzo
E: Erivedge
QD: Daily
QOD: Every other day
M: Monday
W: Wednesday
Th: Thursday
F: Friday

1. A method of treating a cancer associated with a hedgehog (Hh) pathway in a subject in need thereof, the method comprising administering to the subject a therapeutically effective amount of a first antineoplastic agent targeting a component of the Hh pathway, and a therapeutically effective amount of a second antineoplastic agent targeting a component of the Hh pathway, wherein the component of the Hh pathway is selected from Sonic Hedgehog (SHH), a receptor, Patched (PTCH1 and PTCH2), a transmembrane protein, Smoothened (SMO) and gene expression regulators, glioma associated oncogene transcription factors (GLI1, GLI2, GLI3), growth arrest specific protein 1 (GAS1), Cell Adhesion Associated, Oncogene Regulated (CDON), Brother of CDO (BOC), SUFU Negative Regulator Of Hedgehog Signaling (SUFU), Kinesin Family Member 7 (Kif7), hedgehog-interacting protein (HHIP1), and Speckle-Type POZ Protein (SPOP).

2. The method of claim 1, wherein the component of the Hh pathway targeted by the first antineoplastic agent is a component of a first mechanism in the Hh pathway, and the component of the Hh pathway targeted by the second antineoplastic agent is a component of a second mechanism in the Hh pathway, wherein the first mechanism and the second mechanism in the Hh pathway are different.

3. (canceled)

4. The method of claim 2, wherein the first antineoplastic agent targets SMO, and the second antineoplastic agent targets SMO.

5. The method of claim 1, wherein the at least two antineoplastic agents are selected from the group consisting of GDC-0449 (Vismodegib/Erivedge®), Odomzo® (sonidegib, LDE225, Erismodegib), Erivedge® (vismodegib), BMS-833923/XL139, PF-04449913 (Glasdegib), LY2940680 (Taladegib), IPI-926 (Saridegib), Arsenic Trioxide (ATO), Cyclopamine, CUR61414, PF-5274857, TAK-441, MRT-92, Jervine, GANTs, RU-SK/43-129/130, Shh Monoclonal Antibody 5E1-135, and a triazole antifungal agent.

6. The method of claim 1, wherein one of the first and the second antineoplastic agents is a triazole antifungal agent.

7. The method of claim 6, wherein the triazole antifungal agent is selected from itraconazole, fluconazole, posaconazole, voriconazole and any combinations thereof.

8. The method of claim 1, wherein the the first antineoplastic agent comprises itraconazole, and the second antineoplastic agent is selected from sonidegib and vismodegib.

9. The method of claim 8, wherein the itraconazole is administered at a dosage of about 200 mg per day for 2 weeks out of a month and the sonidegib or vismodegib is administered at a dosage of about 100 mg, about 150 mg, or about 200 mg per day, daily or every other day.

10. The method of claim 8, wherein the itraconazole is administered at a dosage of about 100 mg per day for 2 weeks out of a month and the sonidegib or vismodegib is administered at a dosage of about 100 mg, about 150 mg, or about 200 mg per day, daily or every other day.

11. The method of claim 8, wherein the itraconazole is administered for about 8 months at a dosage of about 100 mg per day for 2 weeks out of a month and the sonidegib or vismodegib is administered daily at a dosage of about 100 mg per day, followed by administration of itraconazole of about 100 mg per day for 2 weeks out of a month and administration of sonidegib or vismodegib at a dosage of about 100 mg every other day.

12. (canceled)

13. (canceled)

14. The method of claim 8, wherein the subject was previously treated with vismodegib or sonidegib as a monotherapy and the subject's cancer is resistant, the subject is unresponsive or the subject exhibits side effects to the vismodegib or sonidegib monotherapy.

15. (canceled)

16. (canceled)

17. The method of claim 1, wherein a therapeutically effective dose of each of the at least two antineoplastic agents is reduced relative to the therapeutically effective dose of each antineoplastic agent when administered as a monotherapy.

18. The method of claim 1, wherein the cancer is selected from the group consisting of tumors of skin, brain, muscle, gastrointestinal tract, breast, pancreas, prostate, and central nervous system.

19. The method of claim 1, wherein the cancer is a basal cell carcinoma, advanced/metastatic basal cell carcinoma, or medulloblastoma.

20. (canceled)

21. (canceled)

22. A method of treating a cancer in a subject in need thereof, the method comprising administering to a subject in need thereof an antineoplastic therapy comprising a first antineoplastic agent and a second antineoplastic agent each targeting the Hh pathway, wherein each of the two antineoplastic agents targets a different mechanism of the Hh pathway, and wherein the combined treatment with the first and the second antineoplastic agent overcomes a demonstrated resistance of the cancer to treatment with either the first or the second antineoplastic agent when administered as a monotherapy, or prevents development of resistance of the cancer to treatment with either the first or the second antineoplastic agent when administered as a monotherapy.

23. The method of claim 22, wherein the first antineoplastic agent is vismodegib or sonidegib.

24. The method of claim 23, wherein the second antineoplastic agent is a triazole antifungal agent.

25. (canceled)

26. (canceled)

27. (canceled)

28. (canceled)

29. (canceled)

30. (canceled)

31. (canceled)

32. (canceled)

33. The method of claim 22, wherein the cancer is a basal cell carcinoma, advanced/metastatic basal cell carcinoma, or medulloblastoma.

34. The method of claim 24, wherein the triazole antifungal agent is selected from itraconazole, fluconazole, posaconazole, voriconazole and any combination thereof.

35. The method of claim 34, wherein the triazole antifungal agent comprises itraconazole.
